(19) United States
(12) Patent Application Publication
(10) Pub. No.: US 2024/0311986 A1
(43) Pub. Date: Sep. 19, 2024

(54) METHOD AND APPARATUS FOR GENERATING CONTRAST ENHANCE IMAGE FROM NON-CONTRAST IMAGE USING NEURAL NETWORK MODEL, AND METHOD FOR TRAINNING NEURAL NETWORK MODEL

(71) Applicants: Research & Business Foundation SUNGKYUNKWAN UNIVERSITY, Suwon-si (KR); Samsung Medical Center, Seoul (KR)

(72) Inventors: Simon Sungil WOO, Suwon-si (KR); Jeongho KIM, Suwon-si (KR); Taejune KIM, Suwon-si (KR); Donggeun KO, Suwon-si (KR); Yungyoo LEE, Seoul (KR); Soo Youn HAM, Seoul (KR)

(73) Assignees: Research & Business Foundation SUNGKYUNKWAN UNIVERSITY, Suwon-si (KR); Samsung Medical Center, Seoul (KR)

(21) Appl. No.: 18/603,467

(22) Filed: Mar. 13, 2024

(30) Foreign Application Priority Data
Mar. 13, 2023 (KR) 10-2023-0032398

(51) Int. Cl. G06T 5/94; G06T 5/50; G06T 5/60

(52) U.S. Cl. CPC G06T 5/94; G06T 5/50; G06T 5/60; G06T 2207/10081; G06T 2207/20081; G06T 2207/20084; G06T 2207/20208; G06T 2207/30004

(57) ABSTRACT

There is provided a method and an apparatus capable of generating contrast-enhanced medical images that clearly show contrast-enhanced areas from non-contrast medical images of cross-sections of a patient's body provided by a medical imaging apparatus such as a CT scanner using a neural network model.

### TECHNICAL FIELD

The present disclosure relates to a method and an apparatus for generating contrast-enhanced images from non-contrast images captured by a medical apparatus, such as a CT scanner, based on artificial intelligence.

This work was supported by Institute of Information & communications Technology Planning & Evaluation (IITP) grant funded by Korea government (MSIT) (Research and Development of AI Platforms that Flexibly Adapt to Changes in Privacy Policies and Ensure Compliance with Personal Data Protection Regulations (No. 2022-0-00688) and Development of Advanced Deepfake Detection, Generation Suppression, and Dissemination Prevention Platforms for Countering Malicious Content Manipulation (No. RS-2023-00230337)).

### BACKGROUND

Computed tomography (CT) is capable of obtaining cross-sectional images across a subject's body by imaging the body using a large circular machine with an X-ray generator.

Compared to X-ray imaging apparatus, since CT scanners produce cross-sectional images of organs with low overlap between them, organs and lesions in the organs may be observed more clearly, which has contributed to the wide use of CT scanners for precision examinations of various organs and diseases.

The contrast of the images output from a CT scanner is a crucial factor for accurately diagnosing the subject's lesion. Accordingly, along with the development of CT examination methods, various studies are being conducted to obtain CT images with high contrast.

Conventional methods typically involve administering a contrast agent to the subject to enhance the contrast of CT images. The contrast agent administration method relies on the distribution of blood vessels in the body to increase the contrast of CT images.

For example, lesions commonly exhibit high or low vascular distribution compared to surrounding tissues. Accordingly, administering a contrast agent, which increases X-ray attenuation in the blood vessels, before performing a CT scan enhances the difference in X-ray attenuation between the lesion and its adjacent tissues; thus, visualization of the lesion with enhanced contrast may be obtained from captured images.

However, conventional methods require injection of a significant quantity of contrast agent into the subject, which may cause a harm to the subject. In particular, subjects susceptible to side effects or allergic reactions from the contrast agent may face a greater problem due to the injection of the contrast agent.

### SUMMARY

In view of the above, the present disclosure provides a method and an apparatus capable of generating contrast-enhanced images from non-contrast images captured by a medical apparatus such as a CT scanner based on artificial intelligence.

The aspects of the present disclosure are not limited to the foregoing, and other aspects not mentioned herein will be clearly understood by those skilled in the art from the following description.

In accordance with an aspect of the present disclosure, there is provided a method for generating contrast-enhanced images using a neural network, the method comprises: down-sampling an input non-contrast image to output a down-sampled non-contrast image for each of a plurality of channels; generating a contrast image for each channel corresponding to the down-sampled non-contrast image for each channel using a pre-trained image transformation model; and up-sampling the generated contrast image for each channel to generate a contrast-enhanced image corresponding to the input non-contrast image.

The generating the contrast image for each channel may include extracting a first feature map and a second feature map from the down-sampled non-contrast image for each channel; determining one or more contrast-enhanced areas from the first feature map; and multiplying the second feature map to the one or more contrast-enhanced areas to generate a final feature map for the down-sampled non-contrast image of each channel.

The extracting the first feature map and the second feature map may include extracting the first feature map and the second feature map respectively from the down-sampled non-contrast image for each channel using a plurality of residual blocks having different parameters.

The determining the one or more contrast-enhanced areas may include comparing each of the plurality of feature values of the first feature map with a preconfigured threshold; deriving the one feature value as 0 if one feature value of the plurality of feature values is less than the threshold, or deriving the one feature value as 1 if the one feature value is greater than or equal to the threshold; and determining one or more feature values having a value of 1 among the plurality of feature values as the one or more contrast-enhanced areas.

The generating the contrast image for each channel may further include generating the contrast image for each channel corresponding to the down-sampled non-contrast image for each channel by performing a convolution operation on the final feature map.

The image transformation model may be trained by receiving a training non-contrast image for each channel and a training contrast-enhanced image serving as label data for each channel and transforming the training non-contrast image for each channel to a training contrast image for each image.

The up-sampling the generated contrast image for each channel may include up-sampling the contrast image for each channel to generate a first contrast image for each channel corresponding to the up-sampled contrast image; generating a second contrast image for each channel corresponding to the first contrast image for each channel using the pre-trained image transformation model; and up-sampling the second contrast image for each channel to generate the contrast-enhanced image corresponding to the input non-contrast image using the up-sampled second contrast image.

In accordance with another aspect of the present disclosure, there is provided a contrast-enhanced image generation device using a neural network, the device comprises: a memory configured to store one or more instructions; and a processor configured to execute the one or more instructions stored in the memory, wherein the instructions, when executed by the processor, cause the processor to down-sample an input non-contrast image to output a down-sampled non-contrast image for each of a plurality of channels; generate a contrast image for each channel corresponding to the down-sampled non-contrast image for each channel using a pre-trained image transformation model; and up-sample the generated contrast image for each channel to generate a contrast-enhanced image corresponding to the input non-contrast image.

The processor may be configured to extract a first feature map and a second feature map from the down-sampled non-contrast image for each channel, determine one or more contrast-enhanced areas from the first feature map, and multiply the second feature map to the one or more contrast-enhanced areas to generate a final feature map for the down-sampled non-contrast image of each channel.

The processor may be configured to extract the first feature map and the second feature map respectively from the down-sampled non-contrast image for each channel using a plurality of residual blocks having different parameters.

The processor may be configured to compare each of the plurality of feature values of the first feature map with a preconfigured threshold, derive the one feature value as 0 if one feature value of the plurality of feature values is less than the threshold, or derive the one feature value as 1 if the one feature value is greater than or equal to the threshold, and determine one or more feature values having a value of 1 among the plurality of feature values as the one or more contrast-enhanced areas.

The input non-contrast image and the down-sampled non-contrast image for each channel may include an image capturing an internal organs of a human body, and the one or more contrast-enhanced areas may include an areas in which a vessels depicted in the captured image are located.

The image transformation model is trained by receiving a training non-contrast image for each channel and a training contrast-enhanced image serving as label data for each channel and transforming the training non-contrast image for each channel to a training contrast image for each image.

In accordance with another aspect of the present disclosure, there is provided a method of training a convolutional neural network model, the method comprises: preparing training data including training non-contrast images and training contrast images for a plurality of different color channels; down-sampling the training non-contrast images to output training down-sampled non-contrast images for each different color channel by ; and training an image transformation model to output the respective training contrast images by inputting the respective training down-sampled non-contrast images for each different color channel to the image transformation model.

The training the image transformation model may include extracting a first feature map and a second feature map from the training down-sampled non-contrast images for each different color channel; determining one or more contrast-enhanced areas from the first feature map; and multiplying the second feature map to the one or more contrast-enhanced areas to generate a final feature map for the training down-sampled non-contrast image for each different color channel.

The determining the one or more contrast-enhanced areas may include comparing each of the plurality of feature values of the first feature map with a preconfigured threshold; deriving the one feature value as O if one feature value of the plurality of feature values is less than the threshold, or deriving the one feature value as 1 if the one feature value is greater than or equal to the threshold; and determining one or more feature values having a value of 1 among the plurality of feature values as the one or more contrast-enhanced areas.

The extracting the first feature map and the second feature map may include extracting the first feature map and the second feature map respectively from the training non-contrast image for each different color channel using a plurality of residual blocks having different parameters.

The method may further include generating the respective training contrast images corresponding to the respective training down-sampled non-contrast images by performing a convolution operation on the final feature map.

In accordance with another aspect of the present disclosure, there is provided a non-transitory computer readable storage medium storing computer executable instructions, wherein the instructions, when executed by a processor, cause the processor to perform a method of training a convolutional neural network model, the method comprise: preparing training data including training non-contrast images and training contrast images for a plurality of different color channels; down-sampling the training non-contrast images to output training down-sampled non-contrast images for each different color channel by ; and training an image transformation model to output the respective training contrast images by inputting the respective training down-sampled non-contrast images for each different color channel to the image transformation model.

In accordance with another aspect of the present disclosure, there is provided a computer program stored in a non-transitory computer readable storage medium storing computer executable instructions, wherein the instructions, when executed by a processor, cause the processor to perform a method of training a convolutional neural network model, the method comprise: preparing training data including training non-contrast images and training contrast images for a plurality of different color channels; down-sampling the training non-contrast images to output training down-sampled non-contrast images for each different color channel by ; and training an image transformation model to output the respective training contrast images by inputting the respective training down-sampled non-contrast images for each different color channel to the image transformation model.

The present disclosure may generate and output contrast-enhanced medical images that clearly show contrast-enhanced areas from non-contrast medical images of cross-sections of a patient's body provided by a medical imaging apparatus such as a CT scanner.

Accordingly, the present disclosure may provide a medical staff with contrast-enhanced medical images of the inside of a patient's body even without administering a contrast agent to the patient, eliminating problems stemming from side effects of the contrast agent and improving image interpretation by the medical staff and the accuracy of disease tests for lesions occurring in organs within the patient's body.

### BRIEF DESCRIPTION OF THE DRAWINGS

FIG. 1 shows an apparatus for generating contrast-enhanced images according to an embodiment of the present disclosure.

FIG. 2 conceptually describes the function of a contrast-enhanced image generation program according to one embodiment of the present disclosure.

FIG. 3 conceptually describes the function of a program for generating contrast-enhanced images according to another embodiment of the present disclosure.

FIG. 4 illustrates a method for training an image transformer of FIG. 2.

FIG. 5 illustrates an internal structure of the image transformer of FIG. 2.

FIG. 6 is a flow diagram illustrating a method for generating contrast-enhanced images according to an embodiment of the present disclosure.

FIG. 7 is a flow diagram illustrating a method for generating contrast images of FIG. 6.

FIG. 8 illustrates contrast-enhanced images generated from non-contrast images according to the present disclosure.

### DETAILED DESCRIPTION

The advantages and features of the embodiments and the methods of accomplishing the embodiments will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

Terms used in the present specification will be briefly described, and the present disclosure will be described in detail.

In terms used in the present disclosure, general terms currently as widely used as possible while considering functions in the present disclosure are used. However, the terms may vary according to the intention or precedent of a technician working in the field, the emergence of new technologies, and the like. In addition, in certain cases, there are terms arbitrarily selected by the applicant, and in this case, the meaning of the terms will be described in detail in the description of the corresponding invention. Therefore, the terms used in the present disclosure should be defined based on the meaning of the terms and the overall contents of the present disclosure, not just the name of the terms.

When it is described that a part in the overall specification “includes” a certain component, this means that other components may be further included instead of excluding other components unless specifically stated to the contrary.

In addition, a term such as a “unit” or a “portion” used in the specification means a software component or a hardware component such as FPGA or ASIC, and the “unit” or the “portion” performs a certain role. However, the “unit” or the “portion” is not limited to software or hardware. The “portion” or the “unit” may be configured to be in an addressable storage medium, or may be configured to reproduce one or more processors. Thus, as an example, the “unit” or the “portion” includes components (such as software components, object-oriented software components, class components, and task components), processes, functions, properties, procedures, subroutines, segments of program code, drivers, firmware, microcode, circuits, data, database, data structures, tables, arrays, and variables. The functions provided in the components and “unit” may be combined into a smaller number of components and “units” or may be further divided into additional components and “units”.

Hereinafter, the embodiment of the present disclosure will be described in detail with reference to the accompanying drawings so that those of ordinary skill in the art may easily implement the present disclosure. In the drawings, portions not related to the description are omitted in order to clearly describe the present disclosure.

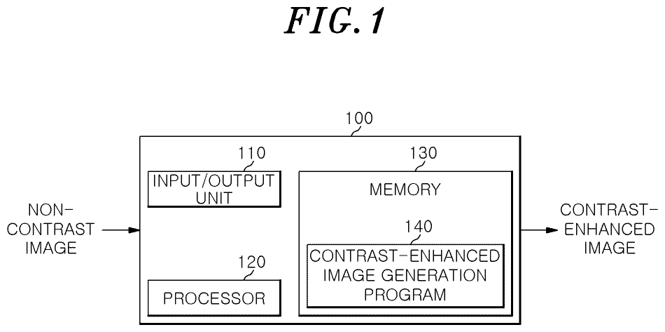

Referring to FIG. 1, the apparatus 100 for generating contrast-enhanced images according to the present embodiment may include an input/output unit 110, a processor 120, and a memory 130.

The input/output unit 110 may receive medical images, for example, medical images of cross-sections of a patient's body, from a medical imaging apparatus (not shown) such as a CT scanner or an MRI scanner. At this time, the received medical images may be non-contrast images, namely, images captured when a contrast agent is not injected to the patient.

Also, the input/output unit 110 may receive an image generated by the processor 120, for example, a contrast-enhanced image corresponding to a previously received non-contrast image, and output the image to an external device, such as a terminal device (not shown) of a medical staff or a patient.

The processor 120 may receive a non-contrast image through the input/output unit 110 and transform the non-contrast image into a contrast-enhanced image using a contrast-enhanced image generation program 140 stored in the memory 130, which will be described later. The processor 120 may output the generated contrast-enhanced image to an external device through the input/output unit 110.

The memory 130 may store the contrast-enhanced image generation program 140 and information necessary for the execution of the program. The contrast-enhanced image generation program 140 may be software that includes instructions for generating a contrast-enhanced image from a non-contrast image received through the input/output unit 110.

Accordingly, the processor 120 may execute the contrast-enhanced image generation program 140 stored in the memory 130 and use the program to generate a contrast-enhanced image from the non-contrast image received through the input/output unit 110.

FIG. 2 conceptually describes the function of a contrast-enhanced image generation program according to one embodiment of the present disclosure.

Referring to FIG. 2, the contrast-enhanced image generation program 140 of the present embodiment may include an encoder 150, an image transformer 160, and a decoder 170.

The encoder 150, the image transformer 160, and the decoder 170 shown in FIG. 2 are conceptually separated for the purpose of describing the functions of the contrast-enhanced image generation program 140, and it should be noted that the present disclosure is not limited to the specific structure.

For example, according to an embodiment of the present disclosure, the functions of the encoder 150, the image transformer 160, and the decoder 170 may be merged or separated or may be implemented as a series of instructions included in one program.

The encoder 150 may down-sample the input non-contrast image and output a non-contrast image for each of a plurality of channels.

The image transformer 160 may generate a contrast image for each of the plurality of channels from a non-contrast image of each of the plurality of channels output from the encoder 150. The image transformer 160 may include a pre-trained neural network model.

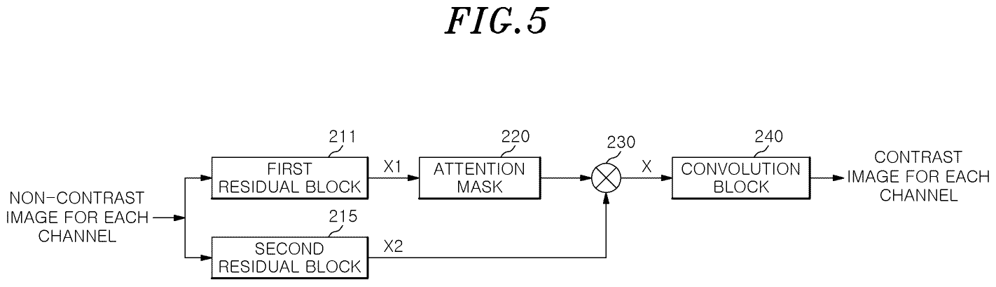

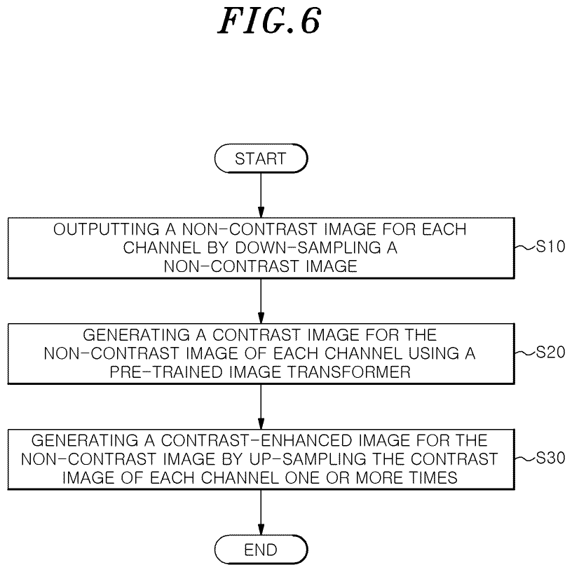

FIG. 4 illustrates a method for training an image transformer of FIG. 2, and FIG. 5 illustrates an internal structure of the image transformer of FIG. 2.

First, referring to FIG. 5, the image transformer 160 according to the present embodiment may include a first residual block 211, a second residual block 215, an attention mask 220, a multiplication block 230, and a convolution block 240.

The first residual block 211 may receive non-contrast images of a first channel among non-contrast images of a plurality of channels from the encoder 150 and extract a first feature map X1 from the non-contrast images of the first channel.

The second residual block 215 may extract a second feature map X2 from the same non-contrast images provided to the first residual block 211, namely, the non-contrast images of the first channel.

Here, the first residual block 211 and the second residual block 215 may have the same structure. It should be noted, however, that since the first residual block 211 and the second residual block 215 have to extract different feature maps from the non-contrast images of the first channel, internal parameter values may be adjusted differently for each residual block.

The first feature map X1 extracted from the first residual block 211 may be a feature map that includes an area which requires contrast enhancement, for example, an area including the blood vessels of the human body, from a non-contrast image of the first channel. Also, the second feature map X2 extracted from the second residual block 215 may be a feature map for the entire area of the non-contrast image of the first channel.

The attention mask 220 may determine one or more contrast-enhanced areas from the first feature map X1 extracted from the first residual block 211.

For example, the attention mask 220 may apply the sigmoid function to the first feature map X1 as shown in Eq. 1 below.

$$sigmoid ( x 1 ) = \frac{1}{1 + {e}^{- x 1}} ∈ ⌈ 0 , 1 ⌉ \qquad [ Eq . 1 ]$$

Also, the attention mask 220 may determine one or more contrast-enhanced areas from the first feature map X1 as shown in Eq. 3 using a step function defined by Eq. 2.

$$f ( x 1 ) = \{ \begin{matrix}1 & ( x 1 > 0 ) \\ 0 & ( x 1 \leq 0 )\end{matrix} \qquad [ Eq . 2 ]$$

$${A}^{ijk} = f ( sigmoid ( X {1}^{ijk} \vert - {θ}_{k} ) - 0.5 ) \qquad [ Eq . 3 ]$$

In Eq. 3, θ represents a predefined threshold.

As described above, among a plurality of feature values of the first feature map X1, the attention mask 220 may derive a feature value exceeding the predefined threshold θ as 1and derive a feature value smaller than or equal to the threshold θ as 0. Accordingly, the attention mask 220 may determine one or more areas with a derived feature value of 1 among the plurality of feature values of the first feature map X1 as the contrast-enhanced area.

As described above, by distinguishing a contrast-enhanced area from a non-contrast area based on the predefined threshold θ in the first feature map X1 of the first channel non-contrast image, the attention mask 220 according to the present embodiment may make the contrast-enhanced area clearly displayed on the contrast image output from the image transformer 160.

The multiplication block 230 multiplies the second feature map X2 extracted from the second residual block 215 to one or more contrast-enhanced areas determined by the attention mask 220 to generate a final feature map X for the first channel non-contrast image.

The convolution block 240 may perform a convolution operation, for example, a 3×3 convolution operation, on the final feature map X output from the multiplication block 230 to output a first channel contrast image for the first channel non-contrast image, namely, a first channel contrast image that includes one or more contrast-enhanced areas.

Also, referring to FIG. 4, the image transformer 160 comprising the first residual block 211, the second residual block 215, the attention mask 220, the multiplication block 230, and the convolution block 240 may be trained by receiving non-contrast images for learning, for example, a non-contrast image for each of a plurality of channels which has passed the encoder 150 and label data serving as transform ground-truth data, for example, an actual contrast-enhanced image corresponding to the non-contrast image for each channel and by transforming the respective non-contrast images to output a contrast image for each channel.

At this time, the image transformer 160 may improve the transform accuracy for the contrast images by repeatedly performing a learning process of transforming input non-contrast images to contrast images while adjusting the internal parameter values to minimize a loss function due to differences between the output contrast images and the corresponding, actual contrast-enhanced images for each channel.

Referring again to FIG. 2, the decoder 170 may generate and output contrast-enhanced images corresponding to the non-contrast images input to the encoder 150 by up-sampling a contrast image for each of a plurality of channels output from the image transformer 160.

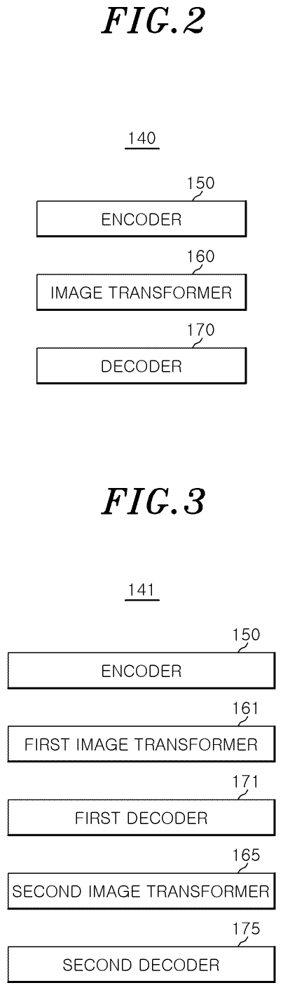

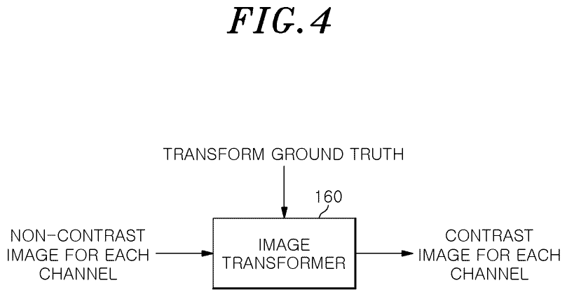

FIG. 3 conceptually describes the function of a program for generating contrast-enhanced images according to another embodiment of the present disclosure.

Referring to FIG. 3, the contrast-enhanced image generation program 141 according to the present embodiment may comprise an encoder 150, a first image transformer 151, a first decoder 171, a second image transformer 165, and a second decoder 175.

The encoder 150 may output a non-contrast image for each of a plurality of channels by down-sampling the input non-contrast images. The encoder 150 may be built on a structure actually the same as that of the encoder 150 described with reference to FIG. 2.

The first image transformer 161 may transform a non-contrast image for each of the plurality of channels output from the encoder 150 to a contrast image for each of the plurality of channels and output the contrast image for each of the plurality of channels.

The first decoder 171 may generate and output a contrast-enhanced image for each of the plurality of channels by up-sampling the contrast image for each of the plurality of channels output from the first image transformer 161.

The second image transformer 165 may again transform the contrast-enhanced image for each of the plurality of channels output from the first decoder 171 to a contrast image for each of the plurality of channels and output the contrast image for each of the plurality of channels.

The second decoder 175 may generate and output contrast-enhanced images corresponding to the non-contrast images input to the encoder 150 by again up-sampling the contrast image for each of the plurality of channels output from the second image transformer 165.

Here, the first image transformer 161 and the second image transformer 165 may have a structure actually the same as that of the image transformer 160 described with reference to

FIG. 2. Also, the first decoder 171 and the second decoder 175 may have a structure actually the same as that of the decoder 170 of FIG. 2.

As described above, the contrast-enhanced image generation program 141 according to the present embodiment may generate a contrast-enhanced image corresponding to the non-contrast image by performing image transformation and up-sampling on the input non-contrast image at least two times.

Accordingly, the contrast-enhanced image generation program 141 according to the present embodiment may improve the performance of contrast-enhanced synthesis for non-contrast images, thereby generating contrast-enhanced images with enhanced contrast between contrast-enhanced areas and the remaining areas from non-contrast images.

As described above, the apparatus 100 for generating contrast-enhanced images according to the present embodiment may generate and output a contrast-enhanced image from a non-contrast image using a pre-trained neural network model.

Accordingly, the present disclosure may provide a medical staff with contrast-enhanced medical images clearly showing contrast-enhanced areas from non-contrast medical images obtained by a medical imaging apparatus such as a CT scanner even without administering a contrast agent to the patient, thereby eliminating problems stemming from side effects of the contrast agent and improving image interpretation by the medical staff and the accuracy of disease tests for lesions occurring in organs within the patient's body.

FIG. 6 is a flow diagram illustrating a method for generating contrast-enhanced images according to an embodiment of the present disclosure.

Referring to FIG. 6, the apparatus 100 for generating contrast-enhanced images may receive medical images, namely, non-contrast images, capturing internal cross-sections of the patient's body from a medical imaging apparatus.

Accordingly, the processor 120 of the apparatus 100 for generating contrast-enhanced images may execute the contrast-enhanced image generation program 140 stored in the memory 130, down-sample non-contrast images through the encoder 150, and output a non-contrast image for each of a plurality of channels S10.

Next, the processor 120 may generate a plurality of contrast images corresponding to the respective non-contrast images for each of the plurality of channels using the pre-trained image transformer 160 S20.

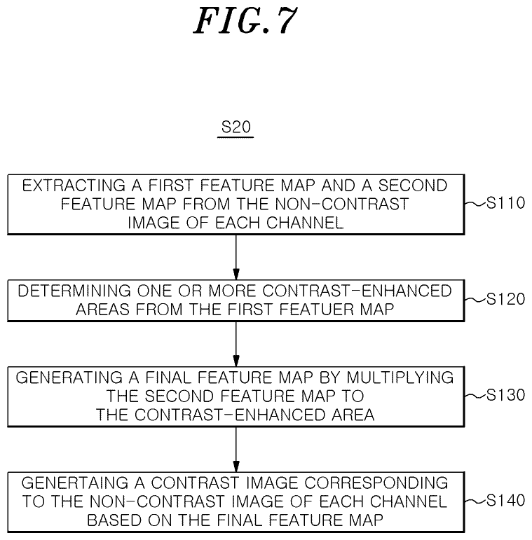

FIG. 7 is a flow diagram illustrating a method for generating contrast images of FIG. 6. Referring to FIGS. 5 and 7, the first residual block 211 of the image transformer 160 may extract a first feature map X1 from a first channel non-contrast image among non-contrast images for each of a plurality of channels, and the second residual block 215 may extract a second feature map X2 from a first channel non-contrast image S110.

Next, the attention mask 220 may determine one or more contrast-enhanced areas from the first feature map using a preconfigured threshold. Then, the multiplication block 230 may multiply the second feature map X2 to one or more contrast-enhanced areas to generate a final feature map X for the first channel non-contrast image.

Next, the convolution block 240 may perform a convolution operation on the final feature map X output from the multiplication block 230 to output a first channel contrast image for the first channel non-contrast image S140.

Referring again to FIG. 6, the decoder 170 may generate and output contrast-enhanced images corresponding to the non-contrast images input to the encoder 150 by up-sampling contrast images for each of a plurality of channels output from the image transformer 160.

Meanwhile, for the convenience of description, the present embodiment assumes that the processor 120 executes the contrast-enhanced image generation program 140 shown in FIG. 2 to generate contrast-enhanced images from non-contrast images input from an external device.

However, the processor 120 may generate contrast-enhanced images from non-contrast images by executing the contrast-enhanced image generation program 141 of FIG. 3. At this time, the generating of the plurality of contrast images and the up-sampling of the plurality of generated contrast images may be performed simultaneously.

For example, the first image transformer 161 of FIG. 3 may transform non-contrast images for each of a plurality of channels provided by the encoder 150 to contrast images for each of the plurality of channels. Subsequently, the first decoder 171 may generate contrast-enhanced images for each of the plurality of channels by up-sampling contrast images for each of the plurality of channels.

Next, the second image transformer 165 may again transform the contrast-enhanced images for each of the plurality of channels to contrast images for each of the plurality of channels, and the second decoder 175 may generate and output contrast-enhanced images corresponding to the non-contrast images by again up-sampling the contrast images for each of the plurality of channels.

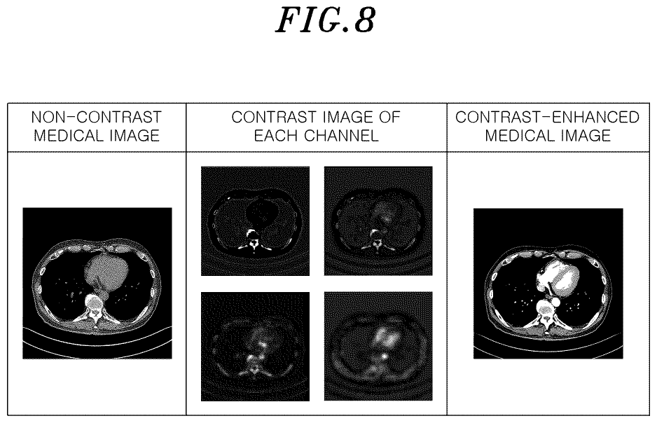

FIG. 8 illustrates contrast-enhanced images generated from non-contrast images according to the present disclosure.

Referring to FIG. 8, when a non-contrast image capturing an internal cross-section of a patient's body is received from an external medical imaging apparatus, the apparatus 100 for generating contrast-enhanced images according to the present embodiment may generate a contrast image for each of a plurality of channels for the non-contrast medical image using a pre-trained neural network model, for example, one or more image transformers 160. Accordingly, the apparatus 100 for generating contrast-enhanced images may generate and output a contrast-enhanced medical image corresponding to the non-contrast medical image by up-sampling the contrast image for each of the plurality of channels.

The contrast-enhanced medical images generated by the apparatus 100 for generating contrast-enhanced images according to the present embodiment may be actually the same as the medical images captured after a contrast agent is injected to the patient.

Therefore, the present disclosure may provide contrast-enhanced medical images with enhanced contrast between contrast-enhanced areas and the remaining areas from non-contrast medical images obtained by a medical imaging apparatus, thereby improving image interpretation by the medical staff and the accuracy of disease tests for lesions occurring in organs within a patient's body.

Combinations of steps in each flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each step of the flowchart. The computer program instructions can also be stored on a computer-usable or computer-readable storage medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each step of the flowchart.

In addition, each step may represent a module, a segment, or a portion of codes which contains one or more executable instructions for executing the specified logical function(s). It should also be noted that in some alternative embodiments, the functions mentioned in the steps may occur out of order. For example, two steps illustrated in succession may in fact be performed substantially simultaneously, or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

What is claimed is:

1. A method for generating contrast-enhanced images using a neural network, the method comprising:
down-sampling an input non-contrast image to output a down-sampled non-contrast image for each of a plurality of channels;
generating a contrast image for each channel corresponding to the down-sampled non-contrast image for each channel using a pre-trained image transformation model; and
up-sampling the generated contrast image for each channel to generate a contrast-enhanced image corresponding to the input non-contrast image.

2. The method of claim 1, wherein the generating the contrast image for each channel includes:
extracting a first feature map and a second feature map from the down-sampled non-contrast image for each channel;
determining one or more contrast-enhanced areas from the first feature map; and
multiplying the second feature map to the one or more contrast-enhanced areas to generate a final feature map for the down-sampled non-contrast image of each channel.

3. The method of claim 2, wherein the extracting the first feature map and the second feature map includes extracting the first feature map and the second feature map respectively from the down-sampled non-contrast image for each channel using a plurality of residual blocks having different parameters.

4. The method of claim 2, wherein the determining the one or more contrast-enhanced areas includes:
comparing each of the plurality of feature values of the first feature map with a preconfigured threshold;
deriving the one feature value as 0 if one feature value of the plurality of feature values is less than the threshold, or deriving the one feature value as 1 if the one feature value is greater than or equal to the threshold; and
determining one or more feature values having a value of 1 among the plurality of feature values as the one or more contrast-enhanced areas.

5. The method of claim 2, wherein the generating the contrast image for each channel further includes generating the contrast image for each channel corresponding to the down-sampled non-contrast image for each channel by performing a convolution operation on the final feature map.

6. The method of claim 1, wherein the image transformation model is trained by receiving a training non-contrast image for each channel and a training contrast-enhanced image serving as label data for each channel and transforming the training non-contrast image for each channel to a training contrast image for each image.

7. The method of claim 1, wherein the up-sampling the generated contrast image for each channel includes:
up-sampling the contrast image for each channel to generate a first contrast image for each channel corresponding to the up-sampled contrast image;
generating a second contrast image for each channel corresponding to the first contrast image for each channel using the pre-trained image transformation model; and
up-sampling the second contrast image for each channel to generate the contrast-enhanced image corresponding to the input non-contrast image using the up-sampled second contrast image.

8. A contrast-enhanced image generation device using a neural network, the device comprising:
a memory configured to store one or more instructions; and
a processor configured to execute the one or more instructions stored in the memory, wherein the instructions, when executed by the processor, cause the processor to
down-sample an input non-contrast image to output a down-sampled non-contrast image for each of a plurality of channels;
generate a contrast image for each channel corresponding to the down-sampled non-contrast image for each channel using a pre-trained image transformation model; and
up-sample the generated contrast image for each channel to generate a contrast-enhanced image corresponding to the input non-contrast image.

9. The contrast-enhanced image generation device of claim 8, wherein the processor is configured to extract a first feature map and a second feature map from the down-sampled non-contrast image for each channel, determine one or more contrast-enhanced areas from the first feature map, and multiply the second feature map to the one or more contrast-enhanced areas to generate a final feature map for the down-sampled non-contrast image of each channel.

10. The contrast-enhanced image generation device of claim 9, wherein the processor is configured to extract the first feature map and the second feature map respectively from the down-sampled non-contrast image for each channel using a plurality of residual blocks having different parameters.

11. The contrast-enhanced image generation device of claim 9, wherein the processor is configured to compare each of the plurality of feature values of the first feature map with a preconfigured threshold, derive the one feature value as 0 if one feature value of the plurality of feature values is less than the threshold, or derive the one feature value as 1 if the one feature value is greater than or equal to the threshold, and determine one or more feature values having a value of 1 among the plurality of feature values as the one or more contrast-enhanced areas.

12. The contrast-enhanced image generation device of claim 9, wherein the input non-contrast image and the down-sampled non-contrast image for each channel include an image capturing an internal organs of a human body, and
wherein the one or more contrast-enhanced areas includes an areas in which a vessels depicted in the captured image are located.

13. The contrast-enhanced image generation device of claim 8, wherein the image transformation model is trained by receiving a training non-contrast image for each channel and a training contrast-enhanced image serving as label data for each channel and transforming the training non-contrast image for each channel to a training contrast image for each image.

14. A method of training a convolutional neural network model to be performed by an electronic device including a memory and a processor, the method comprising:
preparing training data including training non-contrast images and training contrast images for a plurality of different color channels;
down-sampling the training non-contrast images to output training down-sampled non- contrast images for each different color channel by ; and
training an image transformation model to output the respective training contrast images by inputting the respective training down-sampled non-contrast images for each different color channel to the image transformation model.

15. The method of claim 14, wherein the training the image transformation model includes:
extracting a first feature map and a second feature map from the training down-sampled non-contrast images for each different color channel;
determining one or more contrast-enhanced areas from the first feature map; and
multiplying the second feature map to the one or more contrast-enhanced areas to generate a final feature map for the training down-sampled non-contrast image for each different color channel.

16. The method of claim 15, wherein the determining the one or more contrast-enhanced areas includes:
comparing each of the plurality of feature values of the first feature map with a preconfigured threshold;
deriving the one feature value as 0 if one feature value of the plurality of feature values is less than the threshold, or deriving the one feature value as 1 if the one feature value is greater than or equal to the threshold; and
determining one or more feature values having a value of 1 among the plurality of feature values as the one or more contrast-enhanced areas.

17. The method of claim 15, wherein the extracting the first feature map and the second feature map includes extracting the first feature map and the second feature map respectively from the training down-sampled non-contrast image for each different color channel using a plurality of residual blocks having different parameters.

18. The method of claim 15, further comprising generating the respective training contrast images corresponding to the respective training down-sampled non-contrast images by performing a convolution operation on the final feature map.
